(19) United States
(12) Patent Application Publication
(10) Pub. No.: US 2004/0229931 A1
(43) Pub. Date: Nov. 18, 2004

(54) 1-Aryl-3-(indol-5-yl) prop-2-en-1-ones, compositions containing them and use

(75) Inventors: Patrick Mailliet, Fontenay Sous Bois (FR); Fabienne Thompson, Paris (FR); Luc Bertin, Crosnes (FR); Gerard Leclere, Antony (FR); Gilles Tiraboschi, Chevilly (FR)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma S. A., Antony (FR)

(21) Appl. No.: 10/835,631

(22) Filed: Apr. 30, 2004

Related U.S. Application Data
(60) Provisional application No. 60/482,600, filed on Jun. 26, 2003.

(30) Foreign Application Priority Data
Apr. 30, 2003 (FR) 03 05389

(51) Int. Cl. A61K 31/404; C 07D 4 5/02; C07D 209/04

(52) U.S. Cl. 514/414; 514/415; 548/454; 548/510

(57) ABSTRACT

1-Aryl-3-(indol-5-yl)prop-2-en-1-ones, compositions containing them and use thereof are disclosed and claimed. The present invention relates specifically to novel substituted 1-aryl-3-(indol-5-yl)prop-2-en-1-ones of formula (I) having a therapeutic activity, particularly, in oncology.

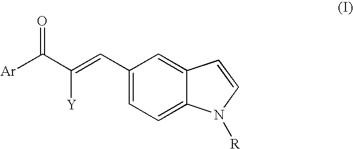

Wherein Ar, Y and R are as described herein.

This application claims the benefit of U.S. Provisional Application No. 60/482,600, filed Jun. 26, 2003 and benefit of priority of French Patent Application No. 03/05,389, filed Apr. 30, 2003.

### BACKGROUND OF THE INVENTION

1. Field of the Invention

The present invention relates to novel chemical compounds, particularly novel 1-aryl-3-(indol-5-yl)prop-2-en-1-ones, the compositions containing them, and their use as medicaments.

More particularly, the invention relates to novel specific 1-aryl-3-(indol-5-yl)-prop-2-en-1-ones exhibiting anticancer activity, and in particular tubulin polymerization inhibiting activity.

2. Description of the Art

1-Aryl-3-(indol-5-yl)prop-2-en-1-ones represent a subclass of the chalcone family in which a phenyl ring has been replaced by an indole. In general, chalcones have been widely described in the literature for more than a century. However, although several hundred publications deal with the therapeutic applications of chalcones, few of them mention their use in oncology.

The following patents and publications may be mentioned among the documents describing the use of chalcones in oncology:

WO 01/72980, discloses substituted chalcones exhibiting anticancer and anti-inflammatory activity.

WO 99/22728, claims in particular, and generically, substituted chalcones, for inhibiting the 5α-reductase activity against steroid hormones, for the treatment of pathologies such as alopaecia, baldness, obesity, skin diseases, prostate cancer and breast cancer.

WO 99/00114 claims the use of chalcones whose prop-2-en-1-one chain may be saturated or unsaturated.

WO 98/58913 discloses chalcones derived from 1-(2-hydroxyphenyl)-3-aryl-prop-2-en-1-one having antiproliferative activity.

EP 288794-B1, claims the use, in oncology, of 1-(aryl)-3-(4-X-phenyl)prop-2-en-1-ones where X represents a substituent NR₂ or NHCOR, with R=alkyl.

WO 91/17749, claims a method for treating cancer using in particular-chalcones. These chalcones are described and claimed very generally.

Michael L. Edwards et al., article which appeared in J. Med. Chem. 1990, vol. 33, pp 1948-1954, present chalcones which can be used as antimitotic agents. Chalcones disclosed therein were tested in vitro on cancer cell lines.

Sylvie Ducki et al., article which appeared in Bioorg. Med. Chem. Letters 1988, vol. 8, pp 1051-1056, present chalcones having an antimitotic activity. Their study is based on the work by Michael L. Edwards et al., cited above. The authors observe that the replacement of a 4-N,N-dimethylamino substituent with 4-methoxy and 3-hydroxy substituents considerably improves the antimitotic activity, in particular in relation to K562 cells.

All of the references listed hereinabove are incorporated herein by reference in their entirety.

However, surprisingly, it has now been found that compounds having the 1-aryl-3-(indol-5-yl)prop-2-en-1-one unit exhibit a high tubulin polymerization inhibiting activity.

Furthermore, these compounds very greatly induce necrosis in vivo, which is a very favorable result for subsequent development of effective drugs for the treatment of cancer.

Next, it was observed that, with the compounds of the present invention, necrosis of the tumor occurs within minutes following the injection of the test product, and that the heart of the tumor is completely destroyed in less than a day, with no apparent effect on neighboring healthy cells. These compounds could therefore be useful for treating patients suffering from inoperable tumors, that is to say whose surgical removal presents a very high risk (i) for the immediate survival of the patient, or (ii) for the possible consequences on their quality of life (invalidation).

Finally, the products of the invention are generally rapidly metabolized by the body, which limits their long-term effect.

Another problem posed consisted in obtaining products having all the advantages described above without the disadvantages frequently encountered during the study of products of the chalcone family. These disadvantages are (i) the problems of cardiac toxicity, which can be evaluated by measuring the inhibition of Herg receptors, (ii) finally, and to a lesser degree, the problems of mutagenicity, which can be evaluated in particular in the Ames test.

### SUMMARY OF THE INVENTION

It should be noted that, up until now, the Applicant has undertaken a major research effort in the abovementioned field and that, to its knowledge, it was only able to identify three very small families of compounds which simultaneously satisfy all these criteria.

These compounds correspond to the following formula (I):

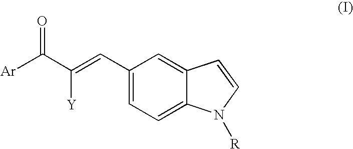

in which:

a) Y is selected from the group consisting of halogen or methyl,

b) Ar is selected from the group consisting of:

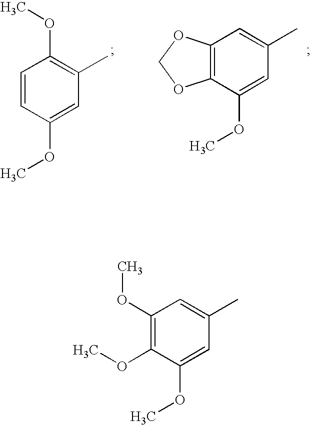

c) R represents:

—CH₂—CH₂—OH.

The compounds according to the invention include the isomers. Among the isomers, the Z isomers and the E isomers form part of the invention. The E isomers are preferred.

Preferably, Y is CH₃.

Finally, within this family, three compounds are distinguishable by their remarkable qualities, in particular because of their additional advantages in relation to problems of solubility, which are usually and unsatisfactorily solved by the preparation of prodrugs or of galenic formulations which can induce major side effects.

These three compounds are:

E-2-methyl-3-[1-(2-hydroxyethyl)-1-H-indol-5-yl]-1-(3,4,5-trimethoxyphenyl)-propenone:

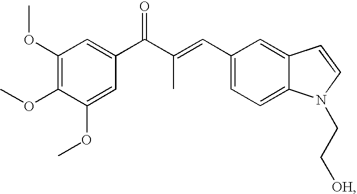

E-2-methyl-3-[1-(2-hydroxyethyl)-1-H-indol-5-yl]-1-(2,5-dimethoxy-phenyl)propenone:

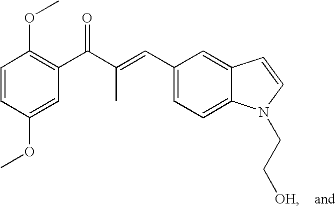

E-2-Methyl-3-[1-(2-hydroxyethyl)indol-5-yl]-1-(3-methoxy-4,5-methylenedioxyphenyl)propenone

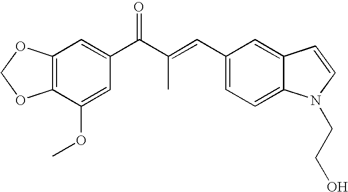

The value of the compounds according to the invention can be advantageously enhanced by their preparation in the form of a pharmaceutical composition, in combination with a pharmaceutically acceptable excipient.

A compound in accordance with the invention may be used for the manufacture of a medicament which is useful for treating a pathological state, in particular a cancer.

The present invention also relates to the therapeutic compositions containing a compound according to the invention, in combination with a pharmaceutically acceptable excipient depending on the mode of administration chosen. The pharmaceutical composition may be provided in solid or liquid form or in the form of liposomes.

### DETAILED DESCRIPTION OF THE INVENTION

Among the solid compositions, there may be mentioned powders, gelatin capsules and tablets. Among the oral forms, there may also be included the solid forms protected against the acidic medium of the stomach. The carriers used for the solid forms consist in particular of inorganic carriers such as phosphates or carbonates, or organic carriers such as lactose, celluloses, starch or polymers. The liquid forms consist of solutions, suspensions or dispersions. They contain, as dispersive carrier, either water, or an organic solvent (ethanol, NMP and the like) or mixtures of surfactants and solvents or of complexing agents and solvents.

The liquid forms will preferably be injectable and will therefore have a formulation acceptable for such a use.

Acceptable routes of administration by injection include the intravenous, intraperitoneal, intramuscular and subcutaneous routes, the intravenous route being preferred.

The dose of compounds of the invention administered will be adjusted by the practitioner according to the route of administration, the patient and the condition of the latter.

The compounds of the present invention may be administered alone or as a mixture with other anticancer agents. Among the possible combinations, there may be mentioned:

alkylating agents and in particular cyclophosphamide, melphalan, ifosfamide, chlorambucil, busulfan, thiotepa, prednimustine, carmustine, lomustine, semustine, steptozotocine, decarbazine, temozolomide, procarbazine and hexamethylmelamine

platinum derivatives such as in particular cisplatin, carboplatin or oxaliplatin

antibiotic agents such as in particular bleomycin, mitomycin, dactinomycin antimicrotubule agents such as in particular vinblastine, vincristine, vindesine, vinorelbine, taxoids (paclitaxel and docetaxel)

anthracyclines such as in particular doxorubicin, daunorubicin, idarubicin, epirubicin, mitoxantrone, losoxantrone

groups I and II topoisomerases such as etoposide, teniposide, amsacrine, irinotecan, topotecan and tomudex

fluoropyrimidines such as 5-fluorouracil, UFT, floxuridine

cytidine analogues such as 5-azacytidine, cytarabine, gemcitabine, 6-mercaptopurine, 6-thioguanine

adenosine analogues such as pentostatin, cytarabine or fludarabine phosphate

methotrexate and folinic acid

various enzymes and compounds such as L-asparaginase, hydroxyurea, trans-retinoic acid, suramin, dexrazoxane, amifostine, herceptin and oestrogen and androgen hormones

antivascular agents such as derivatives of combretastatin or colchicine and their prodrugs.

It is also possible to combine the compounds of the present invention with a radiation treatment. These treatments may be administered simultaneously, separately or sequentially. The treatment will be adapted to the patient to be treated by the practitioner.

A compound in accordance with the invention can promote the disintegration of a cluster of cells derived from a vascular tissue. More particularly, the products of the present invention will be used in their first therapeutic application to inhibit the growth of cancer cells and at the same time the destruction of existing vessels. The inhibition of vascularization is determined by a cell detachment test as described below.

Test Which Makes it Possible to Determine the Inhibition of Vascularization

A test for determining the detachment of endothelial cells was developed in order to select the products with regard to their activity “in vitro”. This test for determining the detachment of endothelial cells is characterized in that the endothelial cells, inoculated into plates whose bottom is covered with a binding agent preferably chosen from gelatin, fibronectin or vitronectin, after culture, are supplemented with a medium containing the compound to be tested, and then the cells are labeled with a fluorescent substance, the cells which have become detached are removed by washing and the fluorescence of the remaining cells is counted in a fluorimeter.

This test consists in measuring the detachment of endothelial cells cultured on substrata based on a binding agent preferably chosen from fibronectin, vitronectin or gelatin. Preferably, a day after the inoculation of the cells in plates containing, for example, 96 wells, the culture medium is replaced with a medium containing the compound to be tested in the absence of serum. The same preparation is prepared six times at three different concentrations (0.1, 0.3 and 0.6 μM) and the control six times without addition of antivascular product. After two hours of treatment with the substance to be tested, the cells are labeled with calcein-AM (1.6 μg/ml) in culture medium supplemented with 0.1% BSA. The cells which have become detached are removed by washing with the culture medium containing 0.1% bovine serum albumin; 100 μl of medium are added to each well. The fluorescence of the remaining cells is counted in a fluorimeter. The data obtained are expressed relative to the control (untreated cells).

The evaluation of the detachment of the endothelial cells in vitro is determined in the following manner. HDMEC cells (Human Dermal Microvascular Endothelial Cells, Promocell, c-122102) are cultured in an ECGM-MV medium which contains 5% foetal calf serum, growth factors (EGF 10 ng/ml, hydrocortisone 1 μg/ml, 0.4% growth supplement with heparin) and antibiotics (amphoteracin 50 ng/ml, gentamycin 50 μg/ml). For the detachment test, the HDMECs are inoculated at 5 000 cells in clear-bottomed 96-well plates (Costar) precoated with fibronectin (10 μg/ml) or vitronectin (1 μg/ml) or gelatin. Twenty-four hours later, the culture medium is replaced with ECGM-MV 0.1% BSA medium containing the products indicated. The concentrations tested are 0.1-0.3 and 1 μM for each product. After two hours of treatment, the cells are labeled for one hour with calcein (1.6 μg/ml, Molecular Probes) in ECGM-MV 0.1% BSA medium. The detached cells are then removed by washing with ECGM-MV 0.1% BSA medium; 100 μl of medium are added to each well. The fluorescence of the cells which remain attached to the substratum of the well is counted using a fluorimeter, Spectrafluor Plus (Tecan excitation 485 nm, and emission 535 nm). The data are the mean of six different samples and are expressed as the percentage of the control (untreated cells).

A cell detachment effect greater than or equal to 15% is considered as significant.

A product in accordance with the invention may be useful for inhibiting the polymerization of tubulin. Inhibition of tubulin may be performed in vitro. An example of a method for inhibiting tubulin in vitro is described below.

Evaluation of the Inhibition of Polymerization of Tubulin

Tubulin is purified from pig brains according to published methods (Shelanski et al., Proc. Natl. Acad. Sci. USA, 70, 765-768. Weingarten et al., 1975, Proc. Natl. Acad. Sci. USA, 72, 1858-1862). Briefly, the brains are ground and centrifuged in an extraction buffer. The tubulin contained in the supernatant of the extract undergoes two successive cycles of polymerization at 37° C. and depolymerization at 4° C., before being separated from the MAPs (Microtubule Associated Proteins) by chromatography on a phosphocellulose P11 column (Whatman). The tubulin thus isolated is more than 95% pure. It is stored in a buffer called RB/2 30% glycerol whose composition is 50 mM MES-NaOH [2-(N-morpholino)ethanesulfonic acid], pH 6.8; 0.25 mM MgCl₂; 0.5 mM EGTA; 30% glycerol (v/v), 0.2 mM GTP (guanosine-5′-triphosphate).

The polymerization of tubulin to microtubules is monitored by turbidimetry as follows: the tubulin is adjusted to a concentration of 10 μM (1 mg/ml) in RB/2 30% glycerol buffer to which 1 mM GTP and 6 mM MgCl₂ are added. The polymerization is triggered by an increase in temperature from 6° C. to 37° C. in a cuvette having an optical path length of 1 cm, placed in a UVIKON 931 spectrophotometer (Kontron) equipped with a thermostated cuvette holder. The increase in the turbidity of the solution is monitored at 350 nm.

The compounds of this invention are dissolved at 10 mM in DMSO and added at variable concentrations (0.5 to 10 μM) to the tubulin solution before polymerization. The IC₅₀ is defined as the concentration of compound which inhibits the rate of polymerization by 50%. A compound whose IC₅₀ is less than or equal to 3 μM is considered as being very active.

Evaluation of the Inhibition of Proliferation of Hela Tumor Cells or of Endothelial Cells HDMEC:

The proliferation of HeLa or HDMEC cells is evaluated by measuring the incorporation of [¹⁴C]thymidine in the following manner. The HeLa cells (epithelial tumor cells of human origin) are cultured in a DMEM medium (Gibco) which contains 10% of foetal calf serum and antibiotics (1% penicillin, 1% streptomycin). To carry out the proliferation test, the cells are inoculated into 96-well Cytostar microplates (Amersham), at the rate of 5000 cells per well. [¹⁴C]Thymidine (0.1 μCi/well) and the products to be evaluated are then added. Variable concentrations of products of up to 10 μM are used; the DMSO (solvent used to solubilize the products) should not exceed 0.5% in the medium. 48 hours after incubation at 37° C., the radioactivity incorporated into the cells is measured by counting the plate in a TR1-LUX counter (Wallac). The IC₅₀ is defined as the concentration of compound which reduces by 50% the radioactivity compared with an untreated control. It is considered that a compound whose IC₅₀ is less than 1 μM is cytotoxic.

Evaluation of Tumor Necrosis In Vivo

Mice are bred either by IFFA-CREDO (Domaine des Oncins, 69210 L'Arbresle, France) from a breed obtained by Jackson Laboratories, Bar Harbor, Me., USA, or alternatively by Charles River France (76410 St Aubin les Elbeuf, France) from a breed obtained by Charles River, USA. The mice initially weigh more than 18 g at the start of the trial. They have free access to food (UAR reference 113, Villemoisson, 91160 Epinay sur Orge, France) and to water.

The tumors used are currently transplanted in our laboratories. All these tumors are at the Frederick Cancer Research Facility (Frederick, Md., USA) at the repository of frozen tumors of the National Cancer Institute (NCI), or at the American Type Culture Collection (ATCC, Rockville, Md., USA).

The techniques for tumor transplantation, chemotherapy and data analysis have been presented in detail (Corbett et al., 1982a; Corbett et al., 1982b).

To summarize, the animals necessary for one experiment are assembled and bilaterally implanted on day 0 (zero).

The growth of solid tumors develops freely up to the desired size. The mice are then treated by intravenous injection of a test compound in solution.

Collection of tumor samples is usually (but not necessarily) performed 24 hours after treatment.

The mice are killed by cerebral dislocation. The implanted tumors, and the skin covering them and the neighboring tissue, are collected and stored in 10% formaldehyde (v/v) (Carlo Erba, Val de Reuil, France).

The samples are then treated, cut into sections, stained with haematoxylin, eosin and saffron yellow, and are then examined macroscopically. The tumor necrosis (necrosis±degeneration) is evaluated microscopically using a scale of extent from 0 to 5:

0=absence of necrosis;

1=minimal, <5%;

2=low, 5-25%;

3=moderate, 25-50%;

4=pronounced, 50-75%;

5=considerable, >75%).

The values assigned to the tumor necrosis 24 hours after administration of the test compound correspond solely to a necrosis depending on the product which can be differentiated with certainty from any existing necrosis resulting from the experiment.

The necrosis due to the experiment was evaluated on an untreated control.

The tumor model is a C51 murine adenocarcinoma. This colon tumor is a grade III mucosal colon adenoma. It is maintained by serial subcutaneous passages every 18 days in female BALB/c mice. The experiments were performed in female BALB/c mice.

Results

Under the conditions as described, the following results were obtained for the example described below:

### EXAMPLE 2

selected dose: 35 mg/kg—grade 5 necrosis

### REFERENCES

CORBETT, T. H., LEOPOLD, W. R., DYKES, D. J., ROBERTS, B. J., GRISWOLD, D. P., Jr. and SCHABEL, F. M., Jr., Toxicity and anticancer activity of a new triazine antifolate (NSC 127755). Cancer Res., 1982a, 42, 1707-1715.

CORBETT, T. H., ROBERTS, B. J., TRADER, M. W., LASTER, W. R., Jr., GRISWOLD, D. P., Jr. and SCHABEL F. M., Jr., Response of transplantable tumors of mice to anthracenedione derivatives alone and in combination with clinically useful agents. Cancer Treat. Rep., 1982b, 66,1187-1200.

Definitions

<<Halogen>> is an element chosen from F, Cl, Br, and I.

The chalcones of general formula (I),

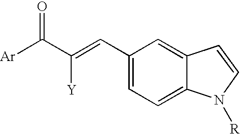

in which Ar is as defined above, Y is different from a halogen atom and R is a protecting group for the NH functional group of the indole, may be prepared by coupling between an aromatic ketone of general formula (II), in which Y is different from a halogen atom, and an aromatic aldehyde of general formula (III), under the conditions described in J. Med. Chem., 1990, 33, 1948, according to scheme (I):

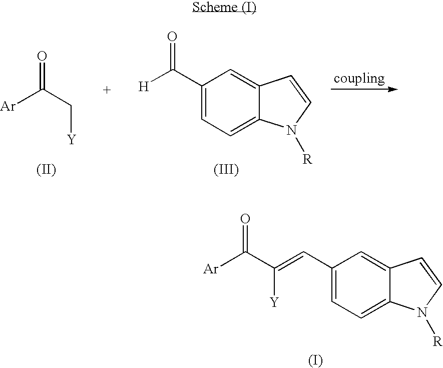

The procedure is generally carried out in a Soxhlet type apparatus at the reflux temperature of an alcohol such as ethanol in the presence of piperidine, acetic acid and molecular sieve.

It is understood that the coupling between the ketone of general formula (II) and the aldehyde of general formula (III) may be carried out with a protecting group such as trimethylsilyl, triethylsilyl or tert-butyidimethylsilyl. The coupling and the cleavage of the protecting group for the NH functional group of the indole may be performed under the conditions described in T. W. Greene, Protective Groups in Organic Chemistry (J. Wiley—Interscience publisher, 1991).

The aromatic aldehydes of general formula (III) are either commercially available or have been previously described in the literature.

The aromatic ketones of general formula (II) are described in the literature and generally prepared from the corresponding aromatic aldehydes which are commercially available. When Y represents a methyl radical, the procedure is advantageously carried out by reacting the aldehyde with a suitably chosen organometallic reagent and then by oxidizing the benzyl alcohol thus obtained under the conditions described in J. Med. Chem., 1990, 33,1948.

The chalcones of general formula (I),

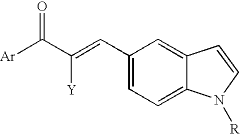

in which Ar is as defined above, R is a substituent which is nonsubstitutable under the conditions for halogen exchange and Y represents a halogen atom, preferably a bromine or chlorine atom, may be prepared by addition of halogen and then dehydrohalogenation of a chalcone in which Y represents a hydrogen atom according to scheme (II). It is particularly advantageous to carry out this addition-elimination sequence on a protected form of the indole nitrogen, for example by a trimethylsilyl, triethylsilyl, tert-butyldimethylsilyl or tert-butyloxycarbonyl, and then to deprotect. Insofar as Y is fluorine, it is not recommended to use silylated protecting groups, as is known by persons skilled in the art.

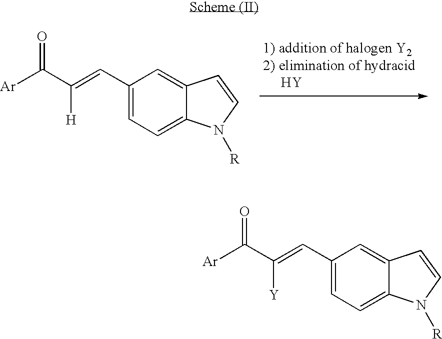

The addition of halogen, preferably bromine or chlorine, is generally carried out in a solvent such as chloroform or carbon tetrachloride, at a temperature between 0 and 50° C. The dehydrohalogenation is generally carried out in a solvent such as dichloromethane, in the presence of an organic or inorganic base such as triethylamine or sodium hydroxide or potassium carbonate, at a temperature between 0° C. and the reflux temperature of the reaction medium. This dehydrohalogenation may be performed simultaneously with the at least partial deprotection of the indole nitrogen, according to the protecting group used, particularly in the case of a tert-butyloxycarbonyl radical.

The chalcones of general formula (I),

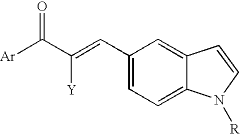

in which Ar, R and Y are in conformity with the invention, may be prepared by alkylation of a chalcone of general formula (I) in which the substituent R is a hydrogen atom. This reaction is performed in two steps. In a first step, after activation of the indole nitrogen in the form of an alkali metal salt, for example with the aid of an alkali metal hydride such as NaH, LiH or KH, an alkyl halide such as 2-bromoethoxy-tert-butyidimethylsilane is caused to react in order to form a product of general formula (I) in which R is 2-tert-butyldimethylsilyloxyethyl. A second step consists in cleaving the trimethylsilyl protective group, as described above, with the aid of a fluoride salt such as tetra-N-butylammonium fluoride, resulting in the formation of a product according to the invention in which R is —CH₂CH₂OH.

The following examples are given by way of illustration of the invention.

### EXAMPLE 1

### E-2-Methyl-3-[1-(2-hydroxyethyl)indol-5-yl]-1-(3,4,5-trimethoxyphenyl)propenone

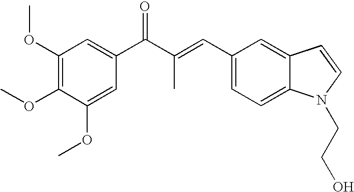

Step 1: 1-(3,4,5-Trimethoxyphenyl)propanone (2.24 g), which may be prepared according to Biorg. Med. Chem. 1998, 8(9), 1051, and 2.76 g of 1-tert-butyloxycarbonylindole-5-carboxaldehyde—which may be prepared according to J. Org. Chem. 2002, 67(17), 6256-59—in 100 ml of ethanol containing 2 ml of piperidine and 1 ml of acetic acid, are successively added to a 25 ml three-necked flask, surmounted with a Soxhlet filled with a 3 Å molecular sieve. The reaction medium is heated under reflux for 48 hours. After cooling, the reaction medium is concentrated under reduced pressure and then taken up in 100 ml of ethyl acetate; the organic phase is washed with water, dried over magnesium sulfate and concentrated under reduced pressure. The crude product is purified by flash chromatography on silica gel (70-230 mesh), eluting with a mixture of cyclohexane and ethyl acetate (70/30 by volume), 2.2 g pure of E-2-methyl-3-[1-(1-tert-butyloxycarbonyl-1-H-indol-5-yl]-1-(3,4,5-trimethoxyphenyl)propenone are obtained in the form of a pale yellow oil which is used as it is in the next step.

Step 2: E-2-Methyl-3-[1-(1-tert-butyloxycarbonyl-1-H-indol-5-yl]-1-(3,4,5-trimethoxyphenyl)propenone (0.7 g) are dissolved in 15 ml of THF. 1.5 ml of methanol and 0.25 g of sodium methoxide are then successively added, and then the reaction medium is stirred for 18 hours at room temperature. After concentrating under reduced pressure, the reaction medium is taken up in 75 ml of ethyl acetate and 35 ml of water. The organic phase is separated by decantation, washed with water, dried over magnesium sulfate and concentrated under reduced pressure. After purification by flash chromatography on silica gel (70-230 mesh), eluting with a mixture of dichloromethane and diisopropyl ether (50/50 by volume), 505 mg of pure E-2-methyl-3-(1-H-indol-5-yl)-1-(3,4,5-trimethoxyphenyl)propenone are obtained in the form of an orange oil whose characteristics are the following:

mass spectrum (EI): m/z=351.

elemental analysis: % C=71.26; % H=6.54; % N=3.72.

Step 3: Sodium hydride (360 mg, at 60% in oil), are slowly added to a solution of 1.4 g of E-2-methyl-3-(1-H-indol-5-yl)-1-(3,4,5-trimethoxyphenyl)-propenone, obtained in step 2, in 40 ml of pyridine, cooled to 0° C. After stirring for 1 hour at 0° C., until the gaseous emission ceases, 960 mg of 2-bromoethoxy-tert-butyldimethylsilane are added and the reaction medium is heated at 60° C. for 2 hours. After addition of 80 ml of water, the reaction medium is extracted 3 times with 50 ml of ethyl acetate. The combined organic phases are washed with water, dried over magnesium sulfate and concentrated to dryness under reduced pressure. The brown oily residue obtained is purified by flash chromatography on silica gel (70-230 mesh), eluting with a mixture of dichloromethane and diisopropyl ether (50/50 by volume). 1.5 g of E-2-methyl-3-[1-(2-tert-butyidimethylsilyloxyethyl)indol-5-yl]-1-(3,4,5-trimethoxyphenyl)propenone are thus obtained in the form of a yellow oil which is used as it is in the next step.

Step 4: A solution of 1.5 g of E-2-methyl-3-[1-(2-tert-butyldimethylsilyloxyethyl)indol-5-yl]-1-(3,4,5-trimethoxyphenyl)propenone in 50 ml of tetrahydrofuran is stirred for 48 hours at room temperature with 23.5 ml of a 1 M solution of tetra-N-butylammonium fluoride in tetrahydrofuran. After addition of 25 ml of water, the reaction medium is extracted 3 times with 5 ml of ethyl acetate. The combined organic phases are washed with water, dried over magnesium sulfate and concentrated to dryness under reduced pressure. The orange residue is purified by flash chromatography on silica gel (70-230 mesh), eluting with a mixture of dichloromethane and ethanol (95/5 by volume), and then by crystallization from isopropanol. 750 mg of E-2-methyl-3-[1-(2-hydroxyethyl)indol-5-yl]-1-(3,4,5-trimethoxyphenyl)propenone are thus obtained in the form of beige crystals whose characteristics are the following:

mass spectrum (EI): m/z=395.

elemental analysis: % C=69.72% H=6.25; % N=3.53.

melting point (Kofler)=119° C.

### EXAMPLE 2

### E-2-Methyl-3-[1-(2-hydroxyethyl)indol-5-yl]-1-(3-methoxy-4,5-methylenedioxyphenyl)propenone

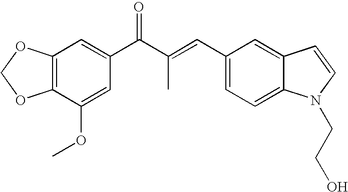

Step 1: In a 250 ml three-neck flask, under an argon atmosphere, 5 g of indole-5-carboxaldehyde are dissolved in 90 ml of DMF and 18 ml of DMSO, then the reaction medium is cooled to 0° C. 2.06 g of sodium hydride at 60% in oil are then added in portions, and then the reaction medium is stirred, while allowing it to return to room temperature until the gaseous emission ceases. 8.6 g of (2-trimethylsilylethyl)oxymethyle chloride are then poured in dropwise, and then the reaction medium is stirred for 20 hours at room temperature. The reaction medium is then poured over a mixture of 300 ml of water and 100 g of crushed ice, and then extracted 3 times with 150 ml of ethyl acetate. The combined organic phases are washed with a saturated aqueous sodium chloride solution, dried over magnesium sulfate and concentrated to dryness under reduced pressure. The brown oil obtained is purified by flash chromatography on silica gel (70-230 mesh), eluting with a mixture of cyclohexane and ethyl acetate (70/30 by volume). 9 g of 1-(2-trimethylsilylethyl)oxymethylindole-5-carboxaldehyde are thus obtained in the form of an orange oil which is used as it is in the next step.

Step 2: 1-(3-Methoxy-4,5-methylenedioxyphenyl)propanone (2.1 g)—which may be prepared according to J. Org. Chem. 1981, 46(14), 2969-71- and 2.76 g of 1-(2-trimethylsilylethyl)oxymethylindole-5-carboxaldehyde in 100 ml of ethanol containing 2 ml of piperidine and 1 ml of acetic acid, are successively added to a 250 ml three-necked flask surmounted with a Soxhlet filled with a 3 Å molecular sieve, while heating under reflux for 96 hours. After cooling, the reaction medium is concentrated under reduced pressure and then taken up in 100 ml of ethyl acetate, and the organic phase is washed with water, dried over magnesium sulfate and concentrated under reduced pressure. The crude product is purified by flash chromatography on silica gel (70-230 mesh), eluting with a mixture of cyclohexane and ethyl acetate (70/30 by volume), and then crystallization from diisopropyl ether. 2 g of pure E-2-methyl-3-[1-(2-trimethylsilylethyl)oxymethyl-1-H-indol-5-yl]-1-(3-methoxy-4,5-methylenedioxyphenyl)propenone are obtained in the form of white crystals whose characteristics are the following:

melting point (Kofler)=90° C.

Step 3: In a 250 ml three-neck flask under an argon atmosphere, 2 g of E-2-methyl-3-[1-(2-trimethylsilylethyl)oxymethyl-1-H-indol-5-yl]-1-(3-methoxy-4,5-methylenedioxyphenyl)propenone are dissolved in 42 ml of THF, and then 4.3 ml of a 1 M solution of tetra-N-butylammonium fluoride in THF are added and the reaction medium is heated under reflux for 20 hours. After concentrating under reduced pressure, the reaction medium is taken up in 75 ml of ethyl acetate and 75 ml of water. The organic phase is separated by decantation, washed with water, dried over magnesium sulfate and concentrated to dryness under reduced pressure. The red oil obtained is purified by par flash chromatography on silica gel (70-230 mesh), eluting with a mixture of cyclohexane and ethyl acetate (70/30 by volume), and then by recrystallization from isopropanol. 420 mg of pure E-2-methyl-3-(1-H-indol-5-yl)-1-(3-methoxy-4,5-methylenedioxyphenyl)propenone are thus obtained in the form of a beige solid whose characteristics are the following:

melting point (Kofler)=140° C.

Step 4: By carrying out the procedure as in step 3 of Example 1, but starting with 671 mg of E-2-methyl-3-(1-H-indol-5-yl)-1-(3-methoxy-4,5-methylenedioxy-phenyl)propenone, obtained in Step 3, Example 2, 180 mg of sodium hydride at 60% in oil and 479 mg of 2-bromoethoxy-tert-butyidimethylsilane in 20 ml of pyridine, there are obtained, after purification by flash chromatography on silica gel (70-230 mesh), eluting with a mixture of ethyl acetate and cyclohexane (30/70 by volume), 830 mg of E-2-methyl-3-[1-(2-tert-butyldimethylsilyloxyethyl)indol-5-yl]-1-(3-methoxy-4,5-methylenedioxy-phenyl)propenone in the form of a yellow oil which is used as it is in the next step.

Step 5: By carrying out the procedure as in step 4 of Example 1, but starting with 830 mg of E-2-methyl-3-[1-(2-tert-butyldimethylsilyloxyethyl)indol-5-yl]-1-(3-methoxy-4,5-methylenedioxyphenyl)propenone in 25 ml of tetrahydrofuran and 13.5 ml of a 1 M solution of tetrabutylammonium fluoride in tetrahydrofuran, for 20 hours, there are obtained, after purification by flash chromatography on silica gel (70-230 mesh), eluting with a mixture of ethyl acetate and cyclohexane (50/50 by volume), and then by recrystallization from isopropyl ether, 385 mg of E-2-methyl-3-[1-(2-hydroxyethyl)indol-5-yl]-1-(3-methoxy-4,5-methylenedioxyphenyl)propenone, in the form of a beige powder whose characteristics are the following:

mass spectrum (EI): m/z=379

elemental analysis: % C=69.84, % H=6.50; % N=3.62.

melting point (Kofler)=83° C.

### EXAMPLE 3

### E-2-Methyl-3-[1-(2-hydroxyethyl)-1-H-indol-5-yl]-1-(2,5-dimethoxyphenyl)propenone

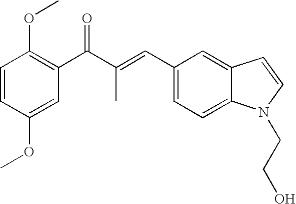

Step 1: 4.86 g of 1-(2,5-dimethoxyphenyl)propanone and 6.13 g of 1-tert-butyloxycarbonylindole-5-carboxaldehyde—which may be prepared according to J. Org. Chem. 2002, 67(17), 6256-59—in 280 ml of ethanol containing 5 ml of piperidine and 2.53 ml of acetic acid, are successively added to a 500 ml three-necked flask, equipped with a Soxhlet filled with a 3 Å molecular sieve, while heating under reflux for 48 hours. After cooling, the reaction medium is concentrated under reduced pressure then taken up in 100 ml of ethyl acetate, and the organic phase is washed with water, dried over magnesium sulfate and concentrated under reduced pressure. The crude product is purified by flash chromatography on silica gel (70-230 mesh), eluting with a mixture of cyclohexane and ethyl acetate (90/10 by volume). 2.3 g of pure E-2-methyl-3-[1-(1-tert-butyloxycarbonyl-1-H-indol-5-yl]-1-(2,5-dimethoxyphenyl)propenone are obtained in the form of a yellow oil which is used as it is in the next step.

Step 2: 2.3 g of E-2-methyl-3-[1-(1-tert-butyloxycarbonyl-1-H-indol-5-yl]-1-(2,5-dimethoxyphenyl)propenone are dissolved in 57 ml of tetrahydrofuran. 5.7 ml of methanol and 0.73 g of sodium methoxide are then successively added, and then the reaction mixture is stirred for 20 hours at room temperature. After concentrating under reduced pressure, the reaction medium is taken up in 75 ml of ethyl acetate and 35 ml of water. The organic phase is separated by decantation, washed with water, dried over magnesium sulfate and concentrated under reduced presssure. After purification by flash chromatography on silica gel (70-230 mesh), eluting with a mixture of ethyl acetate and cyclohexane (30/70 by volume), 1.5 g of pure E-2-methyl-3-(1-H-indol-5-yl)-1-(2,5-dimethoxyphenyl)propenone are obtained in the form of a yellow paste whose characteristics are the following:

mass spectrum (EI): m/z=321.

melting point (Kofler)=52-55° C.

Step 3: By carrying out the procedure as in step 3 of Example 1, but starting with 1 g of E-2-methyl-3-(1-H-indol-5-yl)-1-(2,5-dimethoxyphenyl)propenone, 280 mg of sodium hydride at 60% in oil and 750 mg of 2-bromoethoxy-tert-butyidimethylsilane in 30 ml of pyridine, there are obtained, after purification by flash chromatography on silica gel (70-230 mesh), eluting with a mixture of ethyl acetate and cyclohexane (30/70 by volume), 1.45 g of E-2-methyl-3-[1-(2-tert-butyidimethylsilyloxyethyl)indol-5-yl]-1-(2,5-dimethoxyphenyl)-propenone, in the form of a brown oil which is used as it is in the next step.

Step 4: By carrying out the procedure as in step 4 of Example 1, but starting with 1.45 g of E-2-methyl-3-[1-(2-tert-butyidimethylsilyloxyethyl)indol-5-yl]-1-(2,5-dimethoxyphenyl)propenone in 45 ml of tetrahydrofuran and 25 ml of a 1 M solution of tetra-N-butylammonium fluoride in tetrahydrofuran, for 20 hours, there are obtained, after purification by flash chromatography on silica gel (70-230 mesh), eluting with a mixture of ethyl acetate and cyclohexane (30/70 by volume), and then by recrystallization from isopropanol, 610 mg of E-2-methyl-3-[1-(2-hydroxyethyl)indol-5-yl]-1-(2,5-dimethoxyphenyl)propenone in the form of yellow crystals whose characteristics are the following:

mass spectrum (EI): m/z=365

melting point (Kofler)=112° C.

BIOLOGICAL RESULTS

| | | Tubulin: | Inhibition of cell | | | |
|---|---|---|---|---|---|---|
| | | Inhibition of | proliferation | | | C51 colon |
| Example | | polymerization | IC₅₀ (μM) | | Herg | Tumor necrosis |
| No. | Ames | IC₅₀ (μM) | HDMEC | HeLa | IC₅₀ (μM) | In vivo |
| 1 | negative | 0.8 | 0.00097- | 0,00348- | nd | nd |
| | | | 0.00329 | 0,00375 | | |
| 2 | negative | 0.6 | 0.00022- | 0,00123- | >30 | Grade 5 to 35 |
| | | | 0.00061 | 0,00162 | | mg/kg |
| 3 | negative | 0.83 | nd | nd | nd | nd |

nd: not determined

What is claimed is:

1. A compound of formula (I): 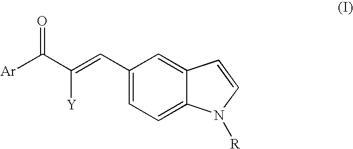
wherein:
a) Y is halogen or methyl;
b) Ar is selected from the group consisting of: 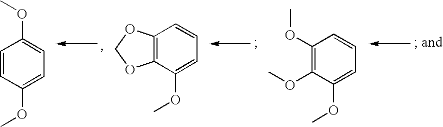
c) R is —CH₂—CH₂—OH;
or a geometrical isomer or a mixture thereof.

2. The compound as set forth in claim 1, wherein Y is CH₃.

3. E-2-Methyl-3-[1-(2-hydroxyethyl)-1-H-indol-5-yl]-1-(3,4,5-trimethoxyphenyl)propenone having the formula:

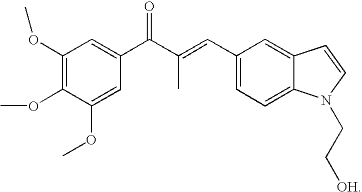

4. E-2-Methyl-3-[1-(2-hydroxyethyl)-1-H-indol-5-yl]-1-(2,5-dimethoxy-phenyl)propenone having the formula:

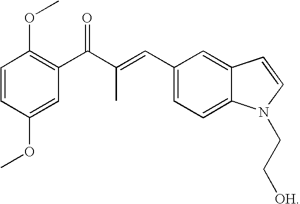

5. E-2-Methyl-3-[1-(2-hydroxyethyl)indol-5-yl]-1-(3-methoxy-4,5-methylenedioxyphenyl)propenone having the formula:

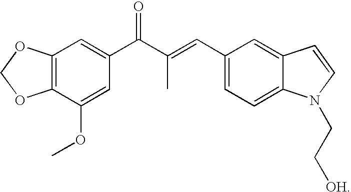

6. A pharmaceutical composition comprising one or more compounds of formula (I) as set forth in claim 1 in combination with a pharmaceutically acceptable excipient.

7. A method of treating a patient suffering from cancer comprising administering to said patient a compound of formula (I) as set forth in claim 1, wherein said compound is having a tubulin polymerization inhibiting activity.

8. The method as set forth in claim 7, wherein said compound of formula (I) is E-2-methyl-3-[1-(2-hydroxyethyl)-1-H-indol-5-yl]-1-(3,4,5-trimethoxyphenyl)propenone.

9. The method as set forth in claim 7, wherein said compound of formula (I) is E-2-methyl-3-[1-(2-hydroxyethyl)-1-H-indol-5-yl]-1-(2,5-dimethoxy-phenyl)propenone.

10. The method as set forth in claim 7, wherein said compound of formula (I) is E-2-methyl-3-[1-(2-hydroxyethyl)indol-5-yl]-1-(3-methoxy-4,5-methylenedioxyphenyl)propenone.

11. A method of treating a patient suffering from cancer comprising administering to said patient a compound of formula (I) as set forth in claim 1, wherein said compound is capable of promoting the detachment of endothelial cells forming the wall of the vessels supplying a tumor.

12. The method as set forth in claim 11, wherein said compound of formula (I) is E-2-methyl-3-[1-(2-hydroxyethyl)-1-H-indol-5-yl]-1-(3,4,5-trimethoxyphenyl)propenone.

13. The method as set forth in claim 11, wherein said compound of formula (I) is E-2-methyl-3-[1-(2-hydroxyethyl)-1-H-indol-5-yl]-1-(2,5-dimethoxy-phenyl)propenone.

14. The method as set forth in claim 11, wherein said compound of formula (I) is E-2-methyl-3-[1-(2-hydroxyethyl)indol-5-yl]-1-(3-methoxy-4,5-methylenedioxyphenyl)propenone.

15. A method of treating a patient suffering from cancer comprising administering to said patient a compound of formula (I) as set forth in claim 1, wherein said compound is capable of promoting tumor necrosis.

16. The method as set forth in claim 15, wherein said compound of formula (I) is E-2-methyl-3-[1-(2-hydroxyethyl)-1-H-indol-5-yl]-1-(3,4,5-trimethoxyphenyl)propenone.

17. The method as set forth in claim 15, wherein said compound of formula (I) is E-2-methyl-3-[1-(2-hydroxyethyl)-1-H-indol-5-yl]-1-(2,5-dimethoxy-phenyl)propenone.

18. The method as set forth in claim 15, wherein said compound of formula (I) is E-2-methyl-3-[1-(2-hydroxyethyl)indol-5-yl]-1-(3-methoxy-4,5-methylenedioxyphenyl)propenone.

19. A method of treating a pathological state in a patient comprising administering to said patient a therapeutically effective amount of a compound of formula (I) as set forth in claim 1.

20. The method as set forth in claim 19, wherein said pathological state is cancer.
